(19) United States
(12) Patent Application Publication
(10) Pub. No.: US 2020/0132628 A1
(43) Pub. Date: Apr. 30, 2020

(54) COMPACT MEASURING APPLIANCE AND METHOD FOR DETECTING HYDROCARBONS

(71) Applicant: BEKO TECHNOLOGIES GMBH, Neuss (DE)

(72) Inventor: Franz-Josef PARUSEL, Langenfeld (DE)

(21) Appl. No.: 16/605,655

(22) PCT Filed: Apr. 23, 2018
(86) PCT No.: PCT/EP2018/060388

§ 371 Date: Oct. 16, 2019

(30) Foreign Application Priority Data
Apr. 21, 2017 (DE) 10 2017 108 609.9
Mar. 9, 2018 (EP) PCT/EP2018/055927

(51) Int. Cl. G01N 27/64; G01N 33/00

(52) U.S. Cl. CPC G01N 27/64; G01N 33/0036; G01N 33/0006; G01N 33/0024

(57) ABSTRACT

A measuring appliance for determining a hydrocarbon concentration in compressed air comprises a housing and the following components arranged in the housing: a main gas line with an inlet for the compressed air, and three additional gas lines, the first branching from the main gas line for compressed air to be measured, the first line connecting the main line to a first switchable valve. The second gas line branches from the main gas line, for reference compressed air, and connects the main gas line to the first switchable valve. The third gas line connects the first switchable valve to a sensor unit and includes copper. A reference gas unit is arranged in the second gas line and contains an oxidation catalyst in which the reference compressed air is produced from the compressed air. A pressure controller is arranged in the main gas line for ensuring a constant through-flow of the compressed air in the range from 3 to 16 bar. The appliance further includes a pressure measuring device arranged upstream of the sensor unit a temperature sensor arranged downstream of the sensor unit, and a cooling apparatus for cooling the reference gas unit.

### TECHNICAL FIELD

The present disclosure relates to a measuring appliance and a method for determining a hydrocarbon concentration in compressed air.

### BACKGROUND

Such measuring appliances with various sensor technologies are known and are used to detect the contact of oil, hydrocarbons and oxidizable gasses, for example, in air or in compressed air. Frequently, for example, electrically heatable semiconductor oxide materials are used, which change their electrical resistance in a heated state depending on the amount of the hydrocarbons contained in the air.

Another method is the detection of hydrocarbons by means of elastomers. To this end, the gas flow to be measured is guided over a body made of heated catalyst material, in the interior space of which a heated platinum spiral is located. The hydrocarbon concentration can be detected via a change in electrical resistance of the heated and of a second platinum spiral, which sets in on the catalyst due to the combustion heat of the hydrocarbon content.

The use of flame ionization detectors is also known. In the case of such devices, the hydrocarbons are incinerated within a gas flow and the voltage change between two electrodes in the flame are measured.

Another method is the detection of hydrocarbon concentration by means of photoionization. Thereby, hydrocarbons are irradiated using ultra-violet light. The amount of energy of the light must be high enough that electrons are driven out of the hydrocarbon. Their number can be measured using electrodes.

The aforementioned methods are suitable, in particular, for the detection of high-level concentrations in oxidizable gasses; however, the detection of low-level concentrations within the low μg/Nm³ range and within the ppb range is not possible in a reliable manner.

The measurement values generated by means of photoionization detectors can only indirectly suggest the measured substance amount since the measurement values also depend on the atomic structure of the compound and can vary quite intensely, even in the case of identical sum formulas. However, provided the compound to be measured is constant, known, as well as uniform to the furthest extent possible, the concentration of the hydrocarbon content can be relatively reliably measured. However, the measurement accuracy drops as the concentration of hydrocarbons decreases. Thereby, in particular, the influence of air-humidity content increases. As the hydrocarbon content decreases, the influence of the air humidity increasingly becomes greater; hydrocarbon content measurements in the lower mg/Nm³, and in particular, in the μg/Nm³ range cannot be carried out in a sufficiently accurate manner.

For various compressed-air applications, different limit values for oil particles are required. Oil particles consist of drop-shaped oil aerosols and oil vapours. Oil aerosols and oil vapours can be partly eliminated or eliminated to a great extent from the compressed-air flow by means of various methods. However, a prompt measurement of oil in compressed air has been an unsolved problem up to this point.

Appliances that are available on the market are often very large, or even divided into two appliance components, a sensor unit and an evaluation unit.

### SUMMARY

The disclosure provides a measuring appliance for determining a hydrocarbon concentration and, if applicable, oxidizable gasses in compressed air that reliably measures extremely low concentrations on the one hand and that does not have the disadvantages of prior art on the other. Furthermore, the disclosure provides a method that is improved with relation to prior art for determining a hydrocarbon concentration and, if applicable, oxidizable gasses in compressed air.

This task is achieved by means of a measuring appliance with the features of Patent Claim 1 and by means of a method with the features of Patent Claim 10. According to the disclosure, the measuring appliance according to the disclosure for determining a hydrocarbon concentration in compressed air has a single housing, in which all components that are crucial to the disclosure are arranged.

Within the context of this disclosure, a differentiation is made between the following compressed-air flows. The term compressed air refers to the compressed air containing hydrocarbons flowing into the measuring appliance. For this purpose, according to the disclosure, the measuring appliance has a main gas line with an inlet. The compressed air containing hydrocarbons can be directly supplied to a sensor unit, subsequently as compressed air to be measured. For this purpose, the measuring appliance has a first gas line branching off from the main gas line, wherein the first gas line connects the main gas line to a first switchable valve. Furthermore, according to the disclosure, the measuring appliance has a second gas line branching off from the main gas line for a reference compressed air. Reference compressed air refers to a compressed air, the hydrocarbon concentration of which is under a detection limit and is therefore zero in terms of the present disclosure. According to the disclosure, the second gas line also connects the main gas line to the first switchable valve. According to the disclosure, a reference gas unit is arranged in the second gas line, said reference gas unit having an oxidation catalyst, which oxidizes the hydrocarbons into carbon dioxide and water. The reference compressed air is thereby supplied to the reference gas unit by branching off the compressed air containing hydrocarbons from the main gas line. According to the disclosure, the first switchable valve is connected to a sensor unit via a third gas line comprising copper.

By means of the fact that the third gas line comprises copper, it is achieved that the respective compressed air flowing through the third gas line assumes the temperature that is predominate inside of the housing or heats up to this.

According to the disclosure, the sensor unit has a photoionization detector with a measurement chamber, in which the hydrocarbon concentration can be detected. The measuring appliance alternately guides the compressed air containing hydrocarbons directly to the sensor unit, which has the photoionization detector, as compressed air to be measured by means of the switchable valve, preferably a solenoid valve, or as reference compressed air via the reference gas unit. A measurement value is determined from a signal difference between the compressed air to be measured and the reference compressed air.

In an alternative embodiment, the reference gas unit can also comprise active carbon or suitable membranes.

In the present document, the measuring principle of the photoionization detector (PID) is based on the ionization of the hydrocarbons present in the gaseous phase by means of UV radiation and the detection of the ionic current resulting therefrom. The intensity of the ionic current is directly proportional to the concentration of the ionized hydrocarbons and, if applicable, other ionizable gasses. The measured signal can be output as a concentration of the measured hydrocarbons in an electronically amplified manner, if applicable.

Favourably, the photoionization detector (PID) indicates the total concentration of all photoionizable compounds contained in the sample and does not differentiate between individual components or substances so that hydrocarbon compounds with less than six carbon atoms (<C6), such as isobutene for example, are detectable.

The reference gas unit integrated in the measuring appliance does not only ensure a regular determination of a new zero point, but is also used for the regular cleaning of the PID by means of the reference compressed air, which is free of hydrocarbons, in particular, being free of oil and grease.

Preferably, this is also supported by a flow-optimized geometry of the measurement chamber. For this purpose, the measurement chamber is particularly preferably funnel-shaped with a diameter that decreases in the flow direction. By means of this, a depositing of molecules, oils and other substances on the inner wall of the measurement chamber is effectively reduced. In addition, it has been shown that a radially orientated inflow of the respective compressed air into the measurement chamber supports the ionization of the hydrocarbons and, if applicable, other gasses due to the dwelling time of the compressed air resulting therefrom.

For example, platinized quartz wool, which is arranged in a reservoir, is used as an oxidation catalyst.

It has been shown that a minimum increase in humidity due to the oxidation in the reference gas unit is negligible if photoionization lamps with 10.6 eV are used in the measurement since, in the case of such measurements, the influence of humidity at its lowest.

Furthermore, the measuring appliance according to the disclosure has a pressure controller arranged in the main gas line for ensuring a constant through-flow of compressed air ranging from 3 to 16 bar so that the same operating conditions for the sensor unit, in particular, the PID, can always be ensured, which, in turn, increases the measurement accuracy.

The detected measurement values are compensated for temperature and pressure for an especially accurate measurement. For this purpose, the measuring appliance according to the disclosure has a pressure measuring appliance arranged upstream of the sensor unit for the determination of a pressure of the respective compressed air flowing into the sensor unit, as well as a temperature sensor arranged downstream of the sensor unit for determining a temperature of the respective compressed air leaving the sensor unit. Thereby, the detected measurement value always refer to a mandatory standard temperature of 20° C. and a mandatory air pressure of 1,000 mbar.

According to the disclosure, the measuring appliance has a cooling device for cooling the reference gas unit arranged within the measuring appliance. In this way, heat developing during operation can be effectively dissipated. Preferably, the cooling device is designed as a fan so that ambient air flows into the housing and heated air flows out of the interior space of the housing.

In a particularly favourable embodiment, for related operational safety, the function of the reference gas unit and the sensor unit, in particular the PID, is continuously monitored and signalled by a display means, preferably an LED. If a defined safety limit is exceeded or exceeded, an alarm is activated, and the user receives the indication that it is necessary to check the appliance. For this purpose, in the case of a functional fault, the LED changes from green to red for example. The through-flow to the sensor unit, in particular, to the PID, is interrupted, whereby the photoionization detector is protected against excessive stress loads.

Preferably, for this purpose, the measuring appliance has a second switchable valve arranged in the third gas line downstream of the first switchable valve, in particular, a solenoid valve, as well as a pressure switch arranged between the second valve and the pressure measuring appliance.

In another favourable embodiment, the measuring appliance has a safety valve arranged in the main gas line downstream of the pressure controller, said safety valve opening at a pressure of ≥4 bar

Preferably, another pressure measuring appliance, for example, a pressure gauge, is arranged in the main gas line downstream of the pressure controller for monitoring the pressure of the compressed air.

Furthermore, the measuring appliance preferably has an integrated evaluation unit, which has evaluation electronics and an operator interface (display). The operating interface can simultaneously be designed as an input unit, for example, by means of a touchscreen.

In the following, the further aspects of the present disclosure will be dealt with in more detail. In order to avoid repeating, reference is made to the aforementioned discussion of the features and advantages of the various embodiments of the measuring appliance according to the disclosure, which are directly applicable to the following discussion of the various embodiment and further embodiments.

In another aspect, the present disclosure relates to a method for determining a hydrocarbon concentration in compressed air by means of the measuring appliance according to the disclosure, comprising the method steps:
guiding the compressed air containing hydrocarbons into the measuring appliance and configuring a constant through-flow at a pressure ranging from 3 to 16 bar,
dividing the compressed air containing hydrocarbons into a compressed air to be measured and a reference compressed air, wherein the compressed air to be measured is applied directly to a sensor unit and the reference compressed air is initially oxidized,
alternately feeding the compressed air to be measured in the reference compressed air into the sensor unit at a defined interval and determining a pressure of the respective compressed air flowing into the sensor unit,
determining a measurement value from a signal difference between the compressed air to be measured and the reference compressed air, and
determining a temperature of the respective compressed air leaving the sensor unit.

According to the disclosure, the compressed air containing the hydrocarbons is continuously divided into the compressed air to be measured and the reference compressed air, wherein the reference compressed air is also continuously supplied to the reference gas unit. If no zero air or reference compressed air is required, this is discharged into the environment via a silencer arranged downstream of the reference unit. If the measuring appliance switches over the measurement of reference compressed air, this is directly available and can be used without any time loss. Thereby, starting-up the reference gas unit like the one required in prior art is no longer required.

According to the disclosure, the calibration of the PID by means of the reference compressed air is carried out according to a defined interval, preferably the compressed air to be measured is supplied to the sensor unit at a factor in the range of 1.5 to 5 times longer than the reference compressed air, preferably 2 to 4 times longer.

For example, in alternation, the compressed air to be measured is supplied for a period of six minutes to the sensor unit and then reference compressed air is supplied to the sensor unit for a period of two minutes. The automatic zero-point comparison compensates for the usual drift effects and thus, even at a low level of hydrocarbon content, in particular oil, for example, being less than 0.01 mg/m³, high-precision readings are detected. Der measurement range is preferably smaller than 2.5 mg/m³, being particularly preferred, at a range of 0.01 to 2.5 mg/m³.

The detected measurement value is a voltage difference between the last measured zero voltage based on the reference compressed air and the currently measured signal voltage of the compressed air to be measured. Preferably, this voltage difference can be compared to stored reference values in order to make a conversion into a ppb concentration possible.

In order to be able to further process the signal voltage of the PID in a processor, it must initially be digitalized. For this, a circuit is used that is referred to as an analogue digital converter. The result is a very precisely measured voltage, which is made available to the processor at certain intervals. The circuit requires at least 0.4 seconds for the most precise conversion possible.

However, since the measurement result is not required at these short intervals, a mean value is preferably formed from a multitude of determined detected measurement values, being particularly preferred, 5 to 15 of them.

Since very low voltages are measured, there is the risk that measurement errors can occur during each and every measurement. By combining a plurality of measurement values, for example 10, the measurement error at a constant signal is three times less than in the individual measurement, i.e. the measurement over four seconds is therefore about three times more accurate than a single measurement value.

Depending on the application, measurement values can also be taken into account for the mean value formation more or less.

It has been shown that, instead of a mean value formation, the determination of a floating mean value leads to better results. Thereby, in addition to the current mean value, preceding mean values will also be taken into consideration. Overall, therefore, the mean value is determined from the current and immediately preceding mean values, wherein the number of included preceding mean values can vary depending on the application.

Due to this favourable calculation method, individual faulty measurements, which are caused by faults in the power supply system for example, can be effectively prevented.

Preferably, the floating mean value is formed from a multitude of mean values, being particularly preferable, 15 to 25 of them.

### BRIEF DESCRIPTION OF THE DRAWINGS

The disclosure is described in the following in detail with reference to the enclosed figures. Thereby, the figures only show favourable embodiments; however, the disclosure should in no way be limited to these. The figures show:

FIG. 1: a schematic diagram of an interior space of an embodiment of the measuring appliance according to the disclosure, and

FIG. 2: a functional diagram or schematic diagram of the fluid flows of the measuring appliance.

### DETAILED DESCRIPTION OF THE DRAWINGS

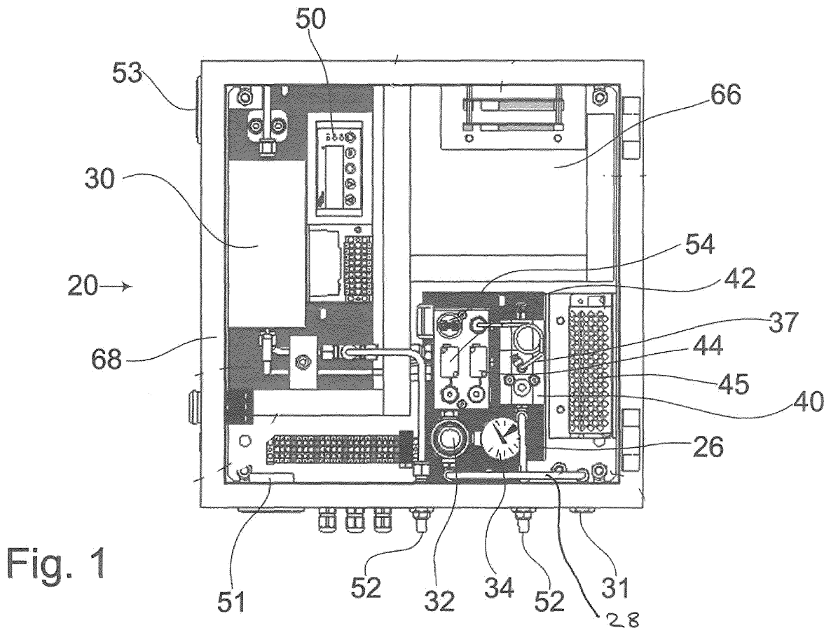

FIG. 1 shows an interior space of an embodiment of the measuring appliance 20 according to the disclosure for the determination of a hydrocarbon concentration in compressed air with all components arranged in a single housing 68.

The measuring appliance 20 has a main gas line 28 with an inlet 31 for the compressed air. A first gas line 38 branches off from the main gas line 28 for compressed air to be measured, wherein the first gas line 38 connects the main gas line 28 to a first switchable valve 42. Furthermore, a second gas line 46 for a reference compressed air branches off from the main gas line 28, wherein the second gas line 46 also connects the main gas line 28 to the first switchable valve 42. Furthermore, the measuring appliance has a third gas line 37 comprising copper connecting the first switchable valve 42 to a sensor unit 26.

The sensor unit 26 has a photoionization detector 40 with a measurement chamber (not shown), to which the respective compressed air is supplied.

Furthermore, a reference gas unit 30 is arranged in the second gas line 46, thereby containing an oxidation catalyst (not shown). The reference compressed air, meaning compressed air free of hydrocarbons, is generated in the reference gas unit 30 from the compressed air containing hydrocarbons. Furthermore, a pressure controller 32 is arranged in the main gas line 28 in order to ensure a constant through-flow of compressed air ranging from 3 to 16 bar.

In order to determine a pressure and a temperature of the compressed air respectively flowing into the sensor unit 26 or flowing out of the sensor unit 26, the measuring appliance 20 has a pressure measurement appliance 56 arranged upstream of the sensor unit 26, as well as a temperature sensor 62 arranged downstream of the sensor unit 26.

Furthermore, the measuring appliance has a cooling device 51, 53 for cooling the reference gas unit 30.

In the third gas line 37, a second switchable valve 44 is arranged downstream of the first switchable valve, 42, and downstream of this, a pressure switch 54 is arranged.

The reference gas unit 30 also has a temperature controller 50 with an alarm function and a cooling device for cooling composed of a fan 51, as well as an air outlet 53.

The compressed air to be measured and the reference compressed air can leave the measuring appliance 20 via silencers 52.

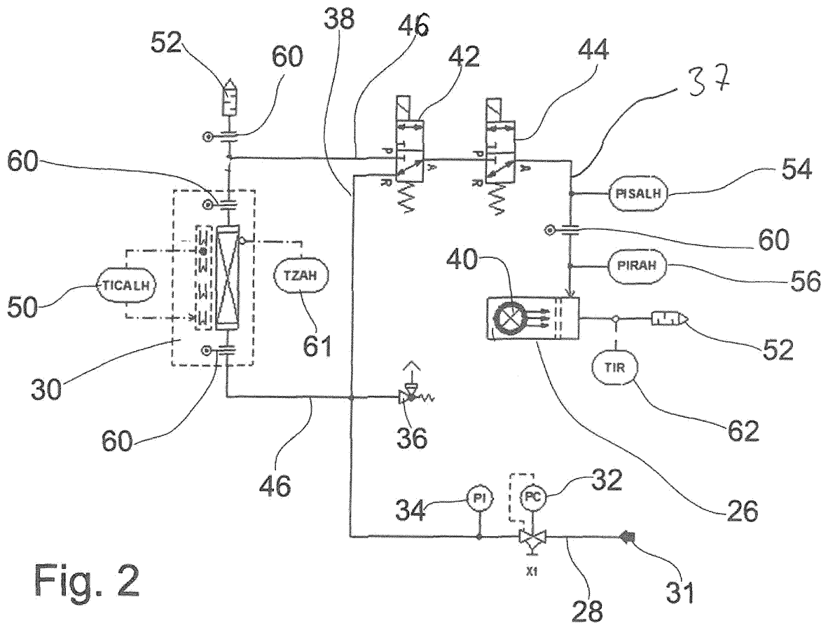

From FIG. 2, the functionality of the measuring appliance 20 according to the disclosure is clarified. The compressed air containing hydrocarbons is guided through the inlet 31 into the measuring appliance 20. The pressure controller 32 regulates the inflow pressure. The safety valve 36, which opens, at a pressure of ≥4 bar for example, follows downstream of the pressure controller 32.

The compressed air to be measured is directly supplied from the compressed air containing hydrocarbons within the main gas line 28 to the sensor unit 26 via the first gas line 38 or to the photoionization detector 40 (PID) via the first switchable valve 42 and the second switchable valve 44.

A reference compressed air is initially supplied to the reference gas unit 30 via a second gas line 46, before this is also supplied to the PID 40 via the two valves 42, 44. Provided that no reference compressed air is required, this is discharged into the environment via a silencer 52.

The reference gas unit 30 has the temperature controller 50. If the temperature of the reference compressed air exceeds a limit value, the second switchable valve 44 is closed and an alarm is issued via an alarm device 61.

Downstream of the two switchable valves 42, 44, the electromechanical pressure switch 54 is arranged, which closes the second switchable valve 44 in the event of overpressure or a negative pressure and triggers an alarm.

The temperature sensor 62 monitors the temperature of the respective compressed air in the sensor unit 26 and outputs an alarm signal upon exceeding a limit value.

Furthermore, chokes 60 are provided within the measuring appliance 20 for regulating the through-flow of the respective compressed air. Furthermore, the measuring appliance 20 has a power supply unit 45 as well as a processor with a related printed circuit board.

1. A measuring appliance for determining a hydrocarbon concentration in compressed air comprising a housing and the following components arranged in the housing, the measuring appliance comprising:
a main gas line with an inlet for the compressed air,
a first gas line branching off from the main gas line for a compressed air to be measured, wherein the first gas line connects the main gas line to a first switchable valve,
a second gas line for a reference compressed air branches off from the main gas line, wherein the second gas line connects the main gas line to the first switchable valve,
a third gas line comprising copper connecting the first switchable valve to a sensor unit, wherein the sensor unit has a photoionization detector with a measurement chamber,
a reference gas unit arranged in the second gas line containing an oxidation catalyst, in which the reference compressed air is generated from the compressed air,
a pressure controller arranged in the main gas line for ensuring a constant through-flow of compressed air ranging from 3 to 16 bar,
a pressure measuring appliance arranged upstream of the sensor unit for determining a pressure of the respective compressed air flowing into the sensor unit,
a temperature sensor arranged downstream of the sensor unit for determining a temperature of the respective compressed air leaving the sensor unit, and
a cooling device for cooling the reference gas unit.

2. The measuring appliance according to claim 1, further comprising a second switchable valve arranged in the third gas line downstream of the first switchable valve.

3. The measuring appliance according to claim 2, wherein the first and the second switchable valve are solenoid valves.

4. The measuring appliance according to claim 2, further comprising a pressure switch arranged in the third gas line between the second valve and the pressure measuring appliance.

5. The measuring appliance according to claim 1, wherein the measurement chamber is funnel-shaped.

6. The measuring appliance according to claim 1, wherein the cooling device for cooling the reference gas unit is configured as a fan so that ambient air flows into the housing and heated air flows out of the interior space of the housing.

7. The measuring appliance according to claim 1, wherein the measuring appliance further includes an evaluation unit.

8. The measuring appliance according to claim 1, comprising a further pressure measuring appliance arranged in the main gas line downstream of the pressure controller.

9. The measuring appliance according to claim 1, comprising a safety belt arranged in the main gas line downstream of the pressure controller, preferably of the further pressure measuring appliance.

10. A method for determining a hydrocarbon concentration in compressed air by means of the measuring appliance according to claim 1, the method including the following steps:
guiding the compressed air containing hydrocarbons into the measuring appliance and configuring a constant through-flow at a pressure ranging from 3 to 16 bar,
dividing the compressed air containing hydrocarbons into a compressed air to be measured and a reference compressed air, wherein the compressed air to be measured is applied directly to a sensor unit and the reference compressed air is initially oxidized,
alternately supplying the compressed air to be measured in the reference compressed air into the sensor unit at a defined interval and determining a pressure of the respective compressed air flowing into the sensor unit,
determining a measurement value from a signal difference between the compressed air to be measured and the reference compressed air, and
determining a temperature of the respective compressed air leaving the sensor unit.

11. The method according to claim 10, wherein the compressed air to be measured is supplied at a factor ranging from 1.5 to 5 times longer than the sensor unit.

12. The method according to claim 10, wherein a mean value is formed from a multitude of detected measurement values.

13. The method according to claim 12, wherein a floating mean value is formed from a multitude of measurement values.
